(19) United States
(12) Patent Application Publication
(10) Pub. No.: US 2018/0141039 A1
(43) Pub. Date: May 24, 2018

(54) ANALYTE DETECTION DEVICES AND SYSTEMS

(71) Applicant: Access Sensor Technologies, Fort Collins, CO (US)

(72) Inventors: Thomas H. Reilly, III, Fort Collins, CO (US); John B. Wydallis, Fort Collins, CO (US)

(21) Appl. No.: 15/820,136

(22) Filed: Nov. 21, 2017

Related U.S. Application Data
(60) Provisional application No. 62/426,062, filed on Nov. 23, 2016.

(51) Int. Cl. B01L 3/00; G01N 33/52; G01N 21/78

(52) U.S. Cl. CPC B01L 3/502; G01N 33/525; G01N 21/78; G01N 2021/7763; B01L 2200/12; B01L 2300/0816; B01L 2300/0887

(57) ABSTRACT

An analyte detection device includes a fluid impermeable layer having a first thickness and at least one inlet port having a diameter, the at least one inlet port defining a fluid pathway through the first thickness. The device also includes a reagent-hosting layer having a second thickness and including at least one of a chemical reagent and a bio-chemical reagent. The reagent-hosting layer is configured to radially receive a sampling fluid via the fluid pathway, where the sampling fluid is configured to interact with the at least one of a chemical reagent and a bio-chemical reagent in the reagent-hosting layer to indicate a characteristic associated with an analyte in the sampling fluid.

### CROSS-REFERENCE TO RELATED APPLICATIONS

This application claims priority to U.S. Provisional Application No. 62/426,062 filed Nov. 23, 2016, entitled “DETECTION DEVICE FOR ANALYSIS OF FLUID PROPERTIES AND METHODS OF USE;” which is hereby incorporated by reference in its entirety for all purposes.

### STATEMENT REGARDING FEDERALLY SPONSORED RESEARCH OR DEVELOPMENT

This invention was made with government support under grant No. 1534786 awarded by the National Science Foundation. The government has certain rights in the invention.

### FIELD

Embodiments of the disclosed subject matter include analyte detection devices. More specifically, embodiments of the disclosure are directed to portable, single use cards for quantifiable analyte detection in a sampling fluid.

### BACKGROUND

Water quality and safety are major concerns for communities. Contaminated water can be global contributors to human disease, disability, and mortality. Waterways are not only used for drinking water, but also for recreation. The safety of waterways, such as rivers, lakes, oceans, and reservoirs, is of importance to water professionals and citizens alike. Spills and contamination of water can include introducing cadmium, lead, iron, zinc, arsenic, copper, and more, all of which are toxic to humans, wildlife, and plants. Damage from spills can be ongoing. Highly chlorinated and therefore corrosive water can cause lead to leach out of pipes and into publicly accessible water sources at unsafe levels. Lead exposure is correlated with severe mental and physical developmental issues, and at very high levels, lead poisoning can result in fatality.

Dissolved aquatic metals are presently measured in two distinct ways: using in-field or laboratory methods. Common laboratory methods include atomic absorption spectroscopy (AAS) and inductively coupled plasma (ICP) coupled with either emissions spectroscopy (OES) or mass spectrometry (MS). These methods are sensitive and selective but require large complicated instruments that limit the total number of samples that can be analyzed and delay the time between sample and results. As a result, in-field assays that are not as sensitive or selective are frequently used.

The in-field analysis technologies can be broken down into three categories: non-instrumented analysis (i.e., dipstick style tests), portable instrument supported analysis, and indirect analysis ‘point and shoot’. Non-instrumented tests include basic visual comparative colorimetric products such as the Hach Colorwheel™, available from Hach of Loveland, Colo.; Millipore Mquant™ and MColortest™, available from Merck KGaA; and CHEMetrics Colorimetrics and Titrimetrics, available from CHEMetrics, Inc. of Midland, Va. These solution chemistry kits require up to 19 steps of adding and mixing reactants which can introduce human error, can have a narrow dynamic range, and are inherently subjective since the result is based on human observation of visual color comparison. The subjectivity can be reduced by adding electronic ‘reading’ of the colorimetric reaction with a fluorimeter or spectrophotometer but these add anywhere from $400 to $3,000. The CHEMetrics Instrumental Colorimeter and Hach SL1000 Portable Parallel Analyzer can streamline analysis by removing the sample preparation and interference blocking steps via disposable Vacu-Vial® and Chemkey® consumables, respectively, but require a relatively high upfront investment.

Point and shoot methods, such as handheld X-Ray Fluorescence (XRF) instruments, are an alternative for solution-phase measurements. XRF instruments are often mentioned synonymously with aquatic testing, but can actually only analyze solids/soil for total elemental components and cannot differentiate between dissolved contaminants (mobile or leachable) and sequestered immobile ones. As many firms now remediate metals via chemical sequestration (i.e., converting the metals to an immobile state and leaving them in the ground), there is a need to differentiate between mobile and immobile metals, which the XRF method does not do. XRF can also be performed off-site, but this requires additional time and cost. Analysis may consume, in some cases, as much as approximately 10% of a project budget, which may amount to significant cost burdens that ultimately create backlogs and costly wait times between sampling and results. The relatively low sample throughput from third-party analyses limits on-site decision making, slows the time-to-completion, and increases overall project costs. A relatively quick, user-friendly, and inexpensive technique to analyze fluids at any location for contaminants such as dissolved metals with on-site, quantifiable results available.

### SUMMARY

Embodiments include a simple, inexpensive, user-friendly, fast, and accurate environmental, chemical, and/or bio-chemical testing device for fluid analysis.

In an Example 1, an analyte detection device, comprises: a fluid impermeable layer having a first thickness and comprising at least one inlet port having a diameter, the at least one inlet port defining a fluid pathway through the first thickness; and a reagent-hosting layer having a second thickness and comprising at least one of a chemical reagent and a bio-chemical reagent, the reagent-hosting layer configured to radially receive a sampling fluid via the fluid pathway, the sampling fluid configured to interact with the at least one of a chemical reagent and a bio-chemical reagent in the porous layer to indicate a characteristic associated with an analyte in the sampling fluid.

In an Example 2, the analyte detection device of Example 1, wherein the characteristic is at least one chosen from a chemical species, a biochemical species, a polarity, a refractive index, an oxidation-reduction potential, a redox activity, a turbidity, a pH, and combinations thereof.

In an Example 3, the analyte detection device of Example 1, wherein the characteristic is the presence or absence of the analyte in the sampling fluid.

In an Example 4, the analyte detection device of any of Examples 1-3, wherein the diameter is greater than or equal to approximately 0.5 mm and less than or equal to approximately 10 mm.

In an Example 5, the analyte detection device of any of Examples 1-4, wherein the second thickness is greater than or equal to approximately 0.05 mm and less than or equal to approximately 2.0 mm.

In an Example 6, the analyte detection device of any of Examples 1-5, wherein the sum of the first thickness and the second thickness is less than or equal to approximately 3.5 mm.

In an Example 7, the analyte detection device of any of Examples 1-6, the porous layer further comprising a detection zone, the detection zone having an additional diameter from greater than or equal to approximately 1 mm and less than or equal to approximately 500 mm.

In an Example 8, the analyte detection device of Example 7, the detection zone indicating the characteristic of the analyte via a color change.

In an Example 9, the analyte detection device of either of Examples 7 or 8, further comprising a central axis wherein the inlet port and the detection zone are concentric about the central axis.

In an Example 10, the analyte detection device of any of Examples 7-9, further comprising a barrier along a circumference of the detection zone, wherein the barrier is continuous or non-continuous.

In an Example 11, the analyte detection device of Example 10, wherein the barrier comprises at least one of a polymer, a glass, a ceramic, a wax, a paint, a cured resin, a plastic, a rubber, a thin film, a metal, a plurality of microparticles, and a plurality of nanoparticles.

In an Example 12, the analyte detection device of any of Examples 1-11, further comprising at least one additional layer, the additional layer having a third thickness, wherein the reagent-hosting layer is disposed between the fluid impermeable layer and the at least one additional layer.

In an Example 13, the analyte detection device of any of Examples 1-12, wherein the reagent-hosting layer comprises at least one of a porous medium and a fibrous medium.

In an Example 14, the analyte detection device of Example 13, wherein the reagent-hosting layer comprises at least one of a paper, a filter paper, a nitrocellulose, a glass fiber mesh, a metal screen, a metal wool, a polymer coated fiber mesh, a polymer filter media, a woven graphite, a non-woven graphite, a carbon fiber mesh, a natural textile, a synthetic textile, a cotton, a wool, and a polyester.

In an Example 15, the analyte detection device of any of Examples 1-14, wherein the fluid impermeable layer is at least partially transparent.

In an Example 16, the analyte detection device of Example 15, wherein the fluid impermeable layer comprises at least one of a polymer, a glass, a ceramic, a wax, a paint, a cured resin, a plastic encasement, a thin film, and a metal coating.

In an Example 17, the analyte detection device of any of Examples 1-16, wherein the at least one of a chemical reagent and a bio-chemical reagent in the porous layer is a material chosen from a redox dye, a solvent polarity dye, a Polypyridyl species, a plurality of nanoparticulates, a thiocarbazone, a glyoxime, a complexometric dye, a pH indicator dye, an Azo dye, and combinations thereof.

In an Example 18, an analyte detection laminate comprises: a central axis; a first layer perpendicular to the central axis and having an inlet aperture, the inlet aperture disposed about the central axis and defining a fluid pathway through the first layer; a second layer, adjacent to the first layer and in fluid communication with the fluid pathway, the second layer comprising: a reagent-hosting medium; at least one of a chemical reagent and a bio-chemical reagent disposed in or on the host medium and configured to interact with a sampling fluid received via the fluid pathway to provide an indicator of a characteristic of an analyte in the sampling fluid; a detection zone, extending radially from the central axis, for quantifying the indicator; and a third layer adjacent to the second layer, wherein the second layer is porous relative to the first and third layers.

In an Example 19, a method of making an analyte detection device comprises: forming a fluid impermeable layer having at least one inlet port defining a fluid pathway therethrough, the at least one inlet port configured to receive a sampling fluid; laminating a reagent-hosting layer to the fluid impermeable layer, the porous layer in fluid communication with the at least one inlet port, the porous layer further comprising a detection zone; and impregnating the detection zone with at least one of a chemical reagent and a bio-chemical reagent, wherein the at least one of a chemical reagent and a bio-chemical reagent is configured to interact with the sampling fluid received at the inlet port and flowing radially in the detection zone to provide an indicator of a characteristic of an analyte in the sampling fluid.

In an Example 20, the method of Example 19, further comprising quantifying the indicator.

While multiple embodiments are disclosed, still other embodiments of the presently disclosed subject matter will become apparent to those skilled in the art from the following detailed description, which shows and describes illustrative embodiments of the disclosed subject matter. Accordingly, the drawings and detailed description are to be regarded as illustrative in nature and not restrictive.

### BRIEF DESCRIPTION OF THE DRAWINGS

FIG. 1 is a top view of an analyte detection device, in accordance with embodiments of the subject matter disclosed herein.

FIG. 2 is an exploded, perspective view of the analyte detection device of FIG. 1, in accordance with embodiments of the subject matter disclosed herein.

FIG. 3A is a perspective view of an analyte detection device with an impermeable layer having a fluid pathway to a porous layer, in accordance with embodiments of the subject matter disclosed herein.

FIG. 3B is a top view of fluid flow through the porous layer of analyte detection device of FIG. 3A, in accordance with embodiments of the subject matter disclosed herein.

FIG. 4A is a perspective view of a porous layer having a barrier, in accordance with embodiments of the subject matter disclosed herein.

FIG. 4B is a perspective view of a porous layer having an alternative barrier, in accordance with embodiments of the subject matter disclosed herein.

FIG. 5 is a top view of an analyte detection device card having more than one detection zone for analysis, in accordance with embodiments of the subject matter disclosed herein.

FIG. 6 is a top view of an analyte detection device card having multiple detection zones for analysis, in accordance with embodiments of the subject matter disclosed herein.

FIG. 7 depicts an illustrative top view of an analyte detection device card 300 configured to facilitate determining a concentration of iron in a sampling fluid, and having a detection zone 302 bounded by a barrier 304, in accordance with embodiments of the subject matter disclosed herein.

FIG. 8 depicts an illustrative graph 400 showing a calibration curve 402 assembled by testing a series of reference solutions to obtain a set of sample points 404 representing the corresponding diameters or color change regions at various iron concentrations, in accordance with embodiments of the subject matter disclosed herein.

FIG. 9 depicts an illustrative graph 400 showing a calibration curve 402 assembled by testing a series of reference solutions to obtain a set of sample points 404 representing the corresponding diameters or color change regions at various iron concentrations, in accordance with embodiments of the subject matter disclosed herein.

While the disclosed subject matter is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

As the terms are used herein with respect to measurements (e.g., dimensions, characteristics, attributes, components, etc.), and ranges thereof, of tangible things (e.g., products, inventory, etc.) and/or intangible things (e.g., data, electronic representations of currency, accounts, information, portions of things (e.g., percentages, fractions), calculations, data models, dynamic system models, algorithms, parameters, etc.), “about” and “approximately” may be used, interchangeably, to refer to a measurement that includes the stated measurement and that also includes any measurements that are reasonably close to the stated measurement, but that may differ by a reasonably small amount such as will be understood, and readily ascertained, by individuals having ordinary skill in the relevant arts to be attributable to measurement error; differences in measurement and/or manufacturing equipment calibration; human error in reading and/or setting measurements; adjustments made to optimize performance and/or structural parameters in view of other measurements (e.g., measurements associated with other things); particular implementation scenarios; imprecise adjustment and/or manipulation of things, settings, and/or measurements by a person, a computing device, and/or a machine; system tolerances; control loops; machine-learning; foreseeable variations (e.g., statistically insignificant variations, chaotic variations, system and/or model instabilities, etc.); preferences; and/or the like.

Although the term “block” may be used herein to connote different elements illustratively employed, the term should not be interpreted as implying any requirement of, or particular order among or between, various blocks disclosed herein. Similarly, although illustrative methods may be represented by one or more drawings (e.g., flow diagrams, communication flows, etc.), the drawings should not be interpreted as implying any requirement of, or particular order among or between, various steps disclosed herein. However, certain embodiments may require certain steps and/or certain orders between certain steps, as may be explicitly described herein and/or as may be understood from the nature of the steps themselves (e.g., the performance of some steps may depend on the outcome of a previous step). Additionally, a “set,” “subset,” or “group” of items (e.g., inputs, algorithms, data values, etc.) may include one or more items, and, similarly, a subset or subgroup of items may include one or more items. A “plurality” means more than one.

As used herein, the term “based on” is not meant to be restrictive, but rather indicates that a determination, identification, prediction, calculation, and/or the like, is performed by using, at least, the term following “based on” as an input. For example, predicting an outcome based on a particular piece of information may additionally, or alternatively, base the same determination on another piece of information.

The terms “up,” “upper,” and “upward,” and variations thereof, are used throughout this disclosure for the sole purpose of clarity of description and are only intended to refer to a relative direction (i.e., a certain direction that is to be distinguished from another direction), and are not meant to be interpreted to mean an absolute direction. Similarly, the terms “down,” “lower,” and “downward,” and variations thereof, are used throughout this disclosure for the sole purpose of clarity of description and are only intended to refer to a relative direction that is at least approximately opposite a direction referred to by one or more of the terms “up,” “upper,” and “upward,” and variations thereof.

### DETAILED DESCRIPTION

Embodiments of the disclosed subject matter include analyte detection systems. Embodiments of an analyte detection system may include an analyte detection device having media hosting a reagent. A sampling fluid may be introduced to the analyte detection device, which facilitates a radial dispersion of the sampling fluid. As the analyte in the sampling fluid encounters the reagent, a color change is produced in a detection zone. The user may determine one or more characteristics of the analyte (and/or the sampling fluid) by measuring a diameter, area, and/or the like, of the color change. In embodiments, the system may also may include, e.g., a ruler, a mobile phone, a computer, a communications network, and/or any number of other devices, users, and/or the like for fluid analysis to provide quantifiable results. Embodiments of the systems and devices disclosed herein may be configured to provide users with quantifiable results within minutes, thus providing information that may, in embodiments, facilitate keeping people and communities safer. Embodiments of the device permit detection and/or characterization of various analytes from various fluid sources on-site and in real time. Fluid samples collected using embodiments of the device can be subsequently processed (e.g., by image analysis) and/or used to generate data regarding fluid quality (e.g., water) and characteristics (e.g., contaminants), which may be used, for example, to establish whether water is safe for drinking, farming, bathing, and/or the like.

Embodiments of the present disclosure take advantage of recent advances in consumer electronics and opensource software platforms. In accordance with these embodiments, the present disclosure provides an analyte detection device that is economical, compact, lightweight, and disposable as compared with conventional devices. Data can be collected manually and/or by using a mobile device application such as, for example, the “Water Chemistry” app (available in the Apple App Store or Google Play), and provided by Access Sensor Technologies, of Fort Collins, Colo., USA.

Throughout this disclosure, the term “radial” and/or “radially” can mean extending along a radius, or extending outwardly from a center point to an arc or segment of a circle (or approximate circle), and/or the like. While this disclosure discusses using the analyte detection device to analyze the presence (and/or absence) of analytes in a sampling fluid such as water, embodiments of the analyte detection device may also, or alternatively, be used to characterize particles in other fluid media including, but not limited to, a liquid, a plasma, and/or the like.

According to embodiments, the analyte detection device includes a fluid impermeable layer having at least one inlet port defined therethrough. The inlet port may be, for example, an aperture through the thickness of the fluid impermeable layer. The inlet port defines a fluid pathway, through the thickness of the fluid impermeable layer to allow a sampling fluid to enter the device. In embodiments, the device further includes a reagent-hosting layer (e.g., a porous layer) adjacent to the fluid impermeable layer. The layers may be placed next to one another directly or otherwise associated with each other via attachment by lamination and/or adhesion. The reagent-hosting layer, which may be a fibrous or non-fibrous (e.g., cellular, etc.) medium, hosts at least one of a chemical reagent and a bio-chemical reagent.

One or more reagents may be loaded onto (and/or into) the reagent-hosting layer. Loading the reagent-hosting layer may include embedding, impregnating, coating, and/or otherwise depositing the reagent-hosting layer with the reagent or reagents. The reagents may be dispersed evenly through the host medium or may be anisotropically distributed. An example of anisotropic distribution may be reagents materially coating the upper and/or lower surface layer of a porous medium.

In operation, a sampling fluid enters the reagent-hosting layer via the inlet port. The reagent-hosting layer receives the sampling fluid via the fluid pathway and the fluid is radially distributed in a detection zone. The sampling fluid interacts with the at least one of a chemical reagent and a bio-chemical reagent in the reagent-hosting layer to indicate a characteristic (e.g., presence or absence, concentration, etc.) of an analyte in the sampling fluid. The indicator may be a visual change such as a color change or other change detectable by the eye or by image analysis techniques known in the art. The concentration of the indicator present, as determined in parts per million (ppm) for example, may be quantified by measuring the diameter (or radius) of the visual indicator in the detection zone. In embodiments, the visual indicator may be measured manually and/or via image analysis for example using a hand held device such as a smart phone equipped with a suitable application.

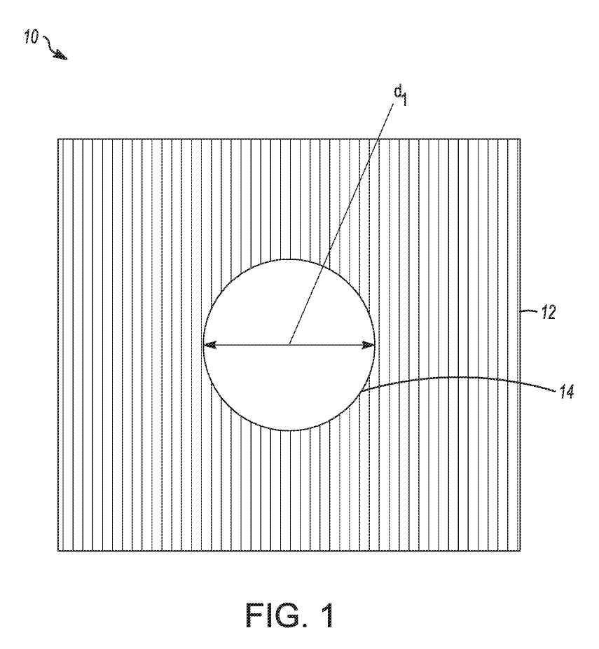

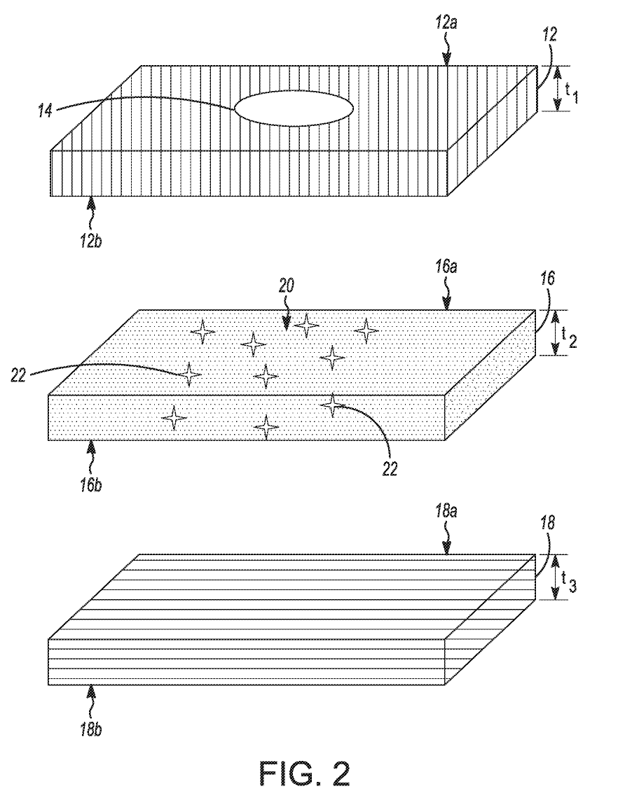

FIG. 1 depicts an illustrative top view of an analyte detection device 10, in accordance with embodiments of the disclosed subject matter; and FIG. 2 depicts an illustrative exploded, perspective view of the analyte detection device 10 of FIG. 1, in accordance with embodiments of the subject matter disclosed herein. As shown, the top layer 12 includes an inlet port 14 having a diameter d₁. The top layer 12 includes a thickness t₁. The thickness t₁ may be any suitable thickness for preventing fluids or moisture from entering the device 10 except via inlet port 14. In embodiments, the thickness t₁ is greater than or equal to approximately 0.05 mm and less than or equal to approximately 1.5 mm. In embodiments, thickness t₁ is approximately 0.05 mm, or approximately 0.10 mm, or approximately 0.2, or approximately 0.3 mm, or approximately 0.4 mm, or approximately 0.5 mm, or approximately 0.6 mm, or approximately 0.7 mm, or approximately 0.8 mm, or approximately 0.9 mm, or approximately 1 mm, or approximately 1.1 mm, or approximately 1.2 mm, or approximately 1.3 mm, or approximately 1.4 mm, or approximately 1.5 mm.

In embodiments, the top layer 12 may be a fluid impermeable layer. The fluid impermeable layer may be formed of any suitable material and the examples herein are non-limiting. In embodiments, for example, the top layer 12 may include at least one material chosen from a polymer, a glass, a ceramic, a wax, a paint, a cured resin, a metal, or combinations thereof. In embodiments, the top layer 12 is at least partially transparent, which means that the fluid impermeable layer 12 may be sufficiently transparent to facilitate optical detection, through the top layer 12 (e.g., by eyesight, camera, etc.) of an indicator of a characteristic of an analyte and/or sampling fluid. In embodiments, the top layer 12 may be or include a colored film or layer to improve optical contrast. Alternatively or additionally, the top layer 12 may include markings on the surface or through the thickness thereby partially obscuring the transparency of the impermeable layer.

The inlet port 14, having diameter d₁, also includes a thickness t₁, which, in the case of the inlet port 14, refers to the depth of the port (corresponding to the thickness of the top layer 12). In embodiments, diameter d₁ is greater than or equal to approximately 0.5 mm and less than or equal to approximately 10 mm. In embodiments, diameter d₁ is approximately 0.5 mm, or approximately 1 mm, or approximately 1.5 mm, or approximately 2 mm, or approximately 2.5 mm, or approximately 3 mm, or approximately 4 mm, or approximately 5 mm, or approximately 6 mm, or approximately 7 mm, or approximately 8 mm, or approximately 9 mm, or approximately 10 mm. In embodiments, diameter d₁ is approximately 2 mm. The inlet port 14 allows fluid to enter the device 10 in a controlled manner.

In embodiments, the inlet port 14 may vary in shape, location, and relative size with respect to the rest of device 10. Shapes and sizes of the inlet port may be varied to influence the direction, flow rate, and/or the two-dimensional or three-dimensional flow pattern of fluid that is delivered to the reagent-hosting layer 16. A multitude of inlet ports may be used rather than a single inlet port. Embodiments of the device 10 include additional inlet ports. In embodiments, the device 10 includes at least one inlet port. In embodiments, the device 10 may include at least two inlet ports, at least three inlet ports, at least four inlet ports, or more. In embodiments, the device 10 may include at least two layers, at least three layers, at least four layers, or more. In embodiments, for example, the device 10 may include at most ten layers.

Disposed adjacent to a lower surface 12b of the top layer 12 is a reagent-hosting layer 16. In embodiments, the reagent-hosting layer 16 may be, include, or be included in a porous medium that allows for radial fluid transport away from the inlet port 14. According to embodiments, the reagent-hosting layer 16 may be porous, fibrous, and/or the like. The reagent-hosting layer 16 includes a thickness t₂ and a detection zone 20. In embodiments, the thickness t₂ is greater than or equal to approximately 0.05 mm and less than or equal to approximately 2.0 mm. In embodiments, thickness t₂ is approximately 0.05 mm, or approximately 0.10 mm, or approximately 0.12, or approximately 0.14 mm, or approximately 0.16 mm, or approximately 0.18 mm, or approximately 0.2 mm, or approximately 0.3 mm, or approximately 0.4 mm, or approximately 0.5 mm, or approximately 1 mm, or approximately 1.5 mm, or approximately 2.0 mm. In embodiments, thickness t₂ is approximately 0.18 mm.

In embodiments, the detection zone 20 is an area and/or volume of the reagent-hosting layer 16 that includes at least one reagent disposed thereon and/or therein. The detection zone 20 may be configured to be analyzed via image analysis to identify characteristics of a sampling fluid and/or one or more analytes therein. The detection zone 20 may include a diameter and/or an area. In embodiments, the detection zone 20 may include at least a portion of the area of the reagent-hosting layer 16. In embodiments, the detection zone 20 may be disposed concentrically about a central axis and may include a diameter d₂. In embodiments, diameter d₂ is greater than or equal to approximately 1 mm and less than or equal to approximately 500 mm. In embodiments, diameter d₂ is greater than or equal to approximately 10 mm and less than or equal to approximately 100 mm. In embodiments, diameter d₂ is greater than or equal to approximately 20 mm and less than or equal to approximately 60 mm. In embodiments, diameter d₂ is approximately 5 mm, or approximately 10 mm, or approximately 20 mm, or approximately 40 mm, or approximately 60 mm, or approximately 80 mm, or approximately 100 mm, or approximately 200 mm, or approximately 300 mm, or approximately 400 mm, or approximately 500. In embodiments, diameter d₂ is approximately 40 mm.

In embodiments, for example, the detection zone 20 may facilitate detecting the presence of an analyte (if any) in the sampling fluid. In embodiments, a characteristic of the analyte (e.g., the presence or absence of the analyte, the concentration of the analyte, etc.) is detected via a color change. Color change is just one example of a visual indicator. The visual indicator may be quantified by manual measurement (e.g., by measuring a radial distance or diameter by ruler or micrometer) and/or via image analysis. According to embodiments, image analysis may be performed using any number of different types of computing devices such as, for example, a laptop, a workstation, a mobile device, and/or the like. In embodiments, for example, an application may be instantiated on a smartphone that utilizes the integrated smartphone camera. Upon opening the application, the user may be prompted, for example, to select an analyte to measure (e.g., via a menu of analytes), a fluid property to measure, and/or the like. In response to selecting an analyte, the application may present a user interface that includes a virtual detection zone overlaying a dynamic image provided by the camera. The user may adjust the smartphone and/or camera such that the detection zone of the device 10 (e.g., as delineated by a visible barrier, a different color, etc.) is completely covered by the virtual detection zone. Upon achieving this positioning, the user may provide an input to the smartphone that causes the smartphone to take a picture, capturing an image of the detection zone of the device 10.

The application (or a related application or application component) may be configured to analyze the image to determine an area of the detection zone that has undergone a color change as a result of an analyte reacting with a reagent. In embodiments, this determination may be made by analyzing the image to identify a region (e.g., an area) of the detection zone that has a different color than the remaining portion of the detection zone. Upon determining the area of color change, the application may be configured to calculate (e.g., based on a determined radius, area, etc.) a characteristic of the analyte and/or sampling fluid. In embodiments, the application may be configured, for example, to measure the area of the color change inside the detection zone and to determine, based on the measured area, the concentration (and/or other characteristic) of the analyte. The application may, for example, present the calculated characteristic to the user such as, for example, by displaying the concentration as parts per million.

In embodiments, this image analysis may be performed using any number of different digital image analysis techniques known to those having skill in the relevant arts. Such techniques may include, for example, edge detection techniques, foreground detection techniques, image segmentation techniques, classifiers, and/or the like. In embodiments, machine learning may be utilized to enhance the accuracy of the image analysis over time. Further, in embodiments, the device 10 may be formed having one or more position indicators (e.g., a dot in the middle of the detection zone, markings disposed around the periphery of the detection zone, etc.) that can be aligned, via the user interface of the application, with corresponding virtual position indicators to ensure proper alignment of the detection zone with the virtual detection zone. In embodiments, image analysis applications may be instantiated on devices separate from the imaging device.

Chemical and/or bio-chemical reagents 22 may be supported by a porous medium of the reagent-hosting layer 16 and may be immobilized or partially immobilized. In embodiments, chemical and/or biochemical reagents 22 may be immobilized using any number of different methods. In embodiments, for example, immobilization may be achieved by selecting reagents largely insoluble in the fluid to be analyzed. For aqueous fluid analysis, reagents may be immobilized by ion-pairing water soluble ionic reagents with water insoluble counter ions. Examples of ion-pairing useful for the invention are combinations of anionic colorimetric reagents such as Zincon (2-Carboxy-2′-hydroxy-5′-sulfoformazyl-benzene monosodium salt), Bromothymol Blue sodium salt (3′,3″-dibromothymolsulfonephthalein sodium salt), Bathophenanthrolinedisulfonic acid disodium salt in combination with hydrophobic cations such as tetrabutylammonium, tetrahexylammonium, tetraoctylamonium ions. These examples are not a complete list of ion-pairing combinations.

Alternatively, ion pairing may be achieved using a hydrophobic (or lipophilic) anion that substantially immobilizes the reagents or the reagent-analyte complex. One non-limiting example of this approach is the use of hexafluorophosphate anions to precipitate the colored divalent iron tris 1,10-phenanthroline complex. This method produces, upon reaction of the reagent with analytes (if present) in the sampling fluid, an example of a visual indicator to provide quantifiable results. Other indicators include any detectable change, such as but not limited to, at least one of a visual change in color or visual indicator, a fluorescence yield, an electrochemical property, an electromagnetic absorbance, an electromagnetic reflectance, an electromagnetic scattering, an electrical conductivity, an acoustic or vibrational impedance, or combinations thereof. As the analytes encounter and react with the reagents loaded in the porous medium they may be largely removed from the fluid. Indicators may represent detected changes in at least one of chemical species, polarity, refractive index, oxidation-reduction potential or redox activity, turbidity, pH, and biochemical species (e.g. enzyme). Reagents deposited on the porous medium may be dispersed evenly throughout or may be anisotropically distributed. An example of anisotropic distribution may be reagents materially coating the upper or lower surface of the porous medium. Reagent distribution may be varied to control the response of the test such that reagents are loaded on the porous medium that vary over the area of the device. A number of reagents may be used in order to detect more than one analyte present in the fluid. These reagents may be combined over the same area (or volumes) or kept separate such that isolated analytes are detected in unique locations on the device.

Reagents useful according to embodiments include reagents to detect changes in at least one of chemical species, polarity, refractive index, oxidation-reduction potential or redox activity, turbidity, pH, and biochemical species (e.g. enzyme). Examples of redox indicator dyes include: 2,6-Dichlorophenolindophenol for vitamin K or vitamin C detection, 3,3′-Dimethylnaphthidine for bromate detection. Examples of solvent polarity dyes include azomerocyanine betaines such as 2,6-Diphenyl-4-(2,4,6-triphenyl-1-pyridinio)phenolate (Reichardt's Dye). Examples of element and species specific colorimetric indicators include: polypyridyl species (e.g. bipyridine, terpyridine and phenanthroline and chemically modified derivatives of those) for the determination of metal ions. Nanoparticulates are useful according to embodiments as direct indicators for refractive index changes. Nanoparticulates with chemically modified surfaces are useful according to embodiments as chemical and biochemical reagents, for example, nanoparticles of gold (Au) and silver (Ag). Examples of reagents suitable for the detection of lead (Pb), zinc (Zn), and cadmium (Cd) include thiocarbazones such as diphenylthiocarbazone. Examples of reagents suitable for the detection of nickel include glyoximes such as dimethylglyoxime. Examples of reagents suitable for the detection of metals include complexometric dyes such as xylenol organge. Examples of reagents suitable for the detection of pH include indicator dyes such as: bromocresol green, phenol red, methyl red. Examples of reagents suitable for the determination of β-glucuronidase enzyme include biochemical enzyme substrates such as 5-Bromo-4-Chloro-3-Indolyl β-D-Glucoronide for the determination of β-glucuronidase enzyme. Examples of reagents suitable for the determination of zinc (Zn) or copper (Cu) include azo dyes such as 2,5-Diphenyl-3-(1-naphthyl) tetrazolium chloride (Tetrazolium Violet) as metabolic indicator, 2-Carboxy-2′-hydroxy-5′-sulfoformazylbenzene monosodium salt (Zincon). Examples of reagents suitable for the detection of organic and metal ion include molecules of a combination of chemical classes such as: 1-(2-Pyridylazo)-2-naphthol and 4-(2-Pyridylazo)resorcinol) as non-selective metal indicators, 2,4,6-Tri(2-pyridyl)-s-triazine for the determination of iron (Fe), 8-Hydroxyquinoline for the determination of aluminum (Al); 1-(4-Nitrophenyl)-3-(4-phenylazophenyl)triazene (Cadion) for determination of cadmium (Cd) and nickel (Ni).

In embodiments, the detection zone 20 may include the entire reagent-hosting layer 16 or a portion of the reagent-hosting layer 16. The detection zone may include any number of different shapes such as, for example, a circular shape, an oval shape, a rectangular shape, and/or the like. In embodiments, the reagent-hosting layer 16 allows for fluid transport through capillary action or by means of an applied force such as elevated pressure at the inlet or a reduced pressure located radially from the inlet 14. The reagent-hosting layer 16 further includes one or more reagents 22 (e.g., chemical reagents and/or bio-chemical reagents) shown schematically throughout thickness t₂ and the detection zone 20 of layer 16. Reagents 22 react with analytes present in a sampling fluid introduced into the reagent-hosting layer 16 via the inlet port 14.

According to embodiments, the reagent-hosting layer 16 may be a porous and/or fibrous medium that is associated with, or otherwise affixed to, the top layer 12. The reagent-hosting layer 16 may be formed of any suitable material and the examples herein are non-limiting. In embodiments, the reagent-hosting layer 16 may include at least one material chosen from a filter paper, a nitrocellulose, a glass fiber mesh, a metal screen, a metal wool, a polymer coated fiber mesh, a polymer filter media, a woven or non-woven graphite, a woven or non-woven carbon fiber mesh, a natural and/or synthetic textile materials such as a cotton, a wool or a polyester, or combinations thereof.

As shown in FIG. 2, the device 10 may include a support layer 18, which includes a thickness t₃. The thickness t₃ may be any suitable thickness for preventing fluids or moisture from entering device 10 except from a lower surface. In embodiments, the thickness t₃ is greater than or equal to approximately 0.05 mm and less than or equal to approximately 1.5 mm. In embodiments, thickness t₃ is approximately 0.05 mm, or approximately 0.10 mm, or approximately 0.2, or approximately 0.3 mm, or approximately 0.4 mm, or approximately 0.5 mm, or approximately 0.6 mm, or approximately 0.7 mm, or approximately 0.8 mm, or approximately 0.9 mm, or approximately 1 mm, or approximately 1.1 mm, or approximately 1.2 mm, or approximately 1.3 mm, or approximately 1.4 mm, or approximately 1.5 mm. The support layer 18 may be fluid impermeable and be configured to support radial fluid flow in the reagent-hosting layer 16. In embodiments, the device 10 may be configured without a support layer 16. In embodiments, the support layer 18 may be a fluid impermeable layer formed of any suitable material and the examples herein are non-limiting. In embodiments, for example, the support layer 18 includes at least one material chosen from a polymer, a glass, a ceramic, a wax, a paint, a cured resin, a metal, or combinations thereof. In embodiments, the top layer 12 and the support layer 18 may be used to bound a specific fluid (e.g. water) while permitting other fluids (e.g. gaseous nitrogen) to pass through unimpeded.

According to embodiments, each layer 12, 16, and 18 includes opposite surfaces referred to herein as upper and lower surfaces, though these terms, as explained above, are used only for clarity of description and are not meant to impart any particular required or desired orientation of the device 10 or its components. In embodiments, one or more of the surfaces of the layers may be at least approximately planar, while, in embodiments, one or more of the surfaces of the layers may be non-planar (e.g., concave, convex, etc.). As shown, the top layer 12 includes an upper surface 12a and a lower surface 12b; the reagent-hosting layer 16 includes an upper surface 16a and a lower surface 16b; and the support layer 18 includes an upper surface 18a and a lower surface 18b.

In embodiments, at least two layers are used to form a laminate. That is, in other words, at least two layers form a laminated structure or material made of layers fixed together to form a detection device. For example, the device 10, also referred to interchangeably herein as a laminate 10 or a card 10, includes at least layer 12 and layer 16. In embodiments, the layers may be fixed together using adhesives applied to at least one of the surfaces 12b, 16a, 16b, 18a, or by other such manner as known to those of skill in the art. In embodiments, for example, the top layer 12 may be affixed to the reagent-hosting layer 16 through permanent (e.g. glue, resin bonding), semi-permanent (e.g. electrostatic cohesion), or temporary means (e.g. external pressure applied to the device to maintain contact). Similarly, the support layer 18 may be affixed to the reagent-hosting layer 16 through permanent (e.g. glue, resin bonding), semi-permanent (e.g. electrostatic cohesion), or temporary means (e.g. external pressure applied to the device to maintain contact).

According to embodiments, a detection device 10 constructed in accordance with embodiments described herein to be a laminate (e.g., card) may be flexible (e.g., much like a laminated piece of paper). In other embodiments, the detection device 10 may be at least partially inflexible, formed using at least one of a rigid top layer 12 and a rigid support layer 18, in which case the layers 12 and 18 form an encasement of the reagent-hosting layer 16. In embodiments, the layers 12 and 18 may be configured to be removably coupled to one another to encase the reagent-hosting layer 16 so that, for example, the encasement can be reused, such as by replacing the reagent-hosting layer 16 with a new and/or different reagent-hosting layer 16 after use.

According to embodiments, the device 10 has dimensions advantageously suited for portability and ease of use. In some embodiments, the device 10 is approximately the same size as (or smaller than) a credit card. In embodiments, the device 10 has a total thickness, t_{T}, which is the sum of the at least two layers adjacently disposed. In embodiments, the total thickness t_{T} is greater than or equal to approximately 0.2 mm and less than or equal to approximately 5.0 mm. In embodiments, the total thickness t_{T} is less than or equal to approximately 3.5 mm, or less than or equal to approximately 4.0, or less than or equal to approximately 4.5 mm, or less than or equal to approximately 5 mm. In embodiments, the device 10 has a shape characterized by a generally flat or planar area, the planar area chosen from at least one of a polygon, a rectangle, a square, a circle, an oval, an ellipse, or a triangle.

The illustrative analyte detection device 10 shown in FIGS. 1 and 2 is not intended to suggest any limitation as to the scope of use or functionality of embodiments of the present disclosure. The illustrative analyte detection device 10 also should not be interpreted as having any dependency or requirement related to any single component or combination of components illustrated therein. Additionally, various components depicted in FIGS. 1 and 2 may be, in embodiments, integrated with various ones of the other components depicted therein (and/or components not illustrated), all of which are considered to be within the ambit of the present disclosure.

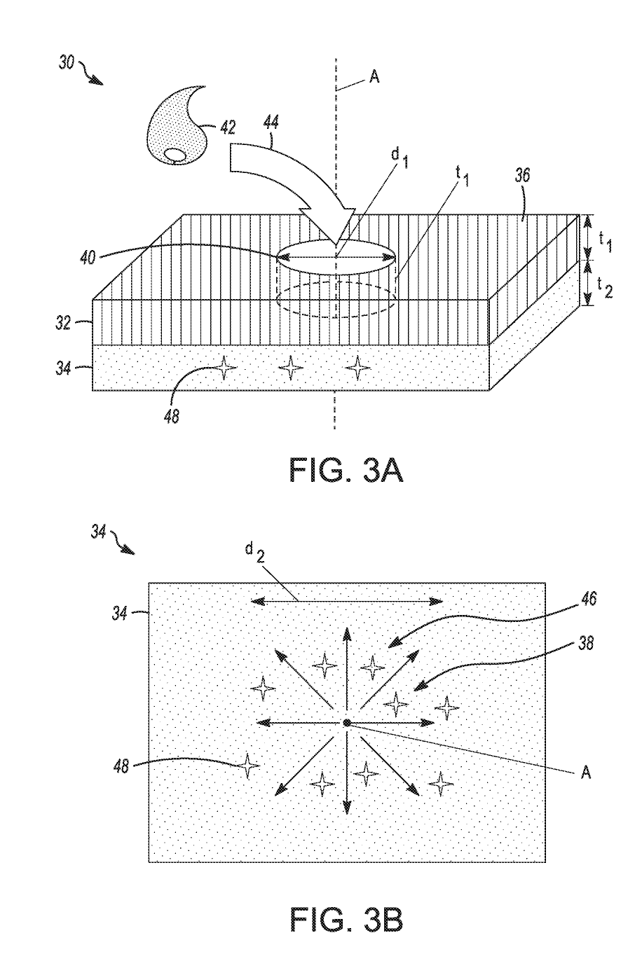

FIG. 3A depicts an illustrative perspective view of an analyte detection device 30, having a top layer 32 (having a thickness t₁) and a reagent-hosting layer 34 (having a thickness t₂) and a central axis A extending through the layers 32 and 34, and at least approximately perpendicular to the upper and lower surfaces of the layers 32 and 34 (e.g., an upper surface 36 of the top layer 32). FIG. 3B depicts an illustrative top view of the reagent-hosting layer 34, showing radial fluid flow 38 through the reagent-hosting layer 34 of the analyte detection device 30 of FIG. 3A, in accordance with embodiments of the subject matter disclosed herein. In embodiments, the device 30 may be similar to the device 10 depicted in FIGS. 1 and 2. The top layer 32 and reagent-hosting layer 34 may be, be similar to, include, or be included in the top layer 12 and reagent-hosting layer 16 depicted in FIGS. 1 and 2.

As shown, the top layer 32 includes an inlet port 40, disposed about axis A and having a diameter d₁, in fluid communication with reagent-hosting layer 34, in accordance with embodiments of the subject matter disclosed herein. In operation, sampling fluid 42 is introduced to the inlet port 40, as illustrated by arrow 44. In embodiments, the fluid 42 is introduced by a pipette or vial (not shown), which may be of the small or miniature, plastic and disposable variety. In embodiments, the pipette or vial has a capacity of about 100 although sampling size may vary according to reagent/analyte combinations and design of analyte detection device. In embodiments, the vial includes a cap or cover. In embodiments, the vial is capped in order to homogenize the sampling fluid by shaking or other means. Alternatively in embodiments, the inlet port 40 may be exposed to the sampling fluid 42 by dipping the device 30 into a sampling fluid 42. In embodiments, for example, the device 30 may be dipped into the sampling fluid for a pre-determined time (e.g., a few seconds) or until a detection zone 46 of the reagent-hosting layer 34 absorbs as much of the sampling fluid 42 that it can. When using dipping as a method of exposure to sampling fluid, a third impermeable support layer (e.g., the support layer 18 depicted in FIG. 2) may be utilized.

As shown in FIG. 3B, directional fluid flow 38 extends radially from axis A outward through the reagent-hosting layer 34. In embodiments, directional fluid flow 38, referred to interchangeably herein as radial fluid flow 38, may travel through the reagent-hosting layer 34 thickness t₂. In embodiments, the top layer 32 may be configured to radially receive sampling fluid 42 via the inlet port 40 to extend outwardly from the inlet port 40 in the detection zone 46. As the sampling fluid 42 flows radially outward from the inlet port 40, it reacts with one or more reagents 48 disposed within the detection zone 46, causing a color change in the detection zone 46, which indicates a characteristic of an analyte in the sampling fluid. A characteristic of an analyte may include the presence or absence of the analyte in the sampling fluid, an amount (or concentration) of analyte in the sampling fluid, the type of analyte, and/or the like. In embodiments, reagents may additionally or alternatively be used to indicate one or more characteristics of the sampling fluid such as, for example, polarity, a refractive index, an electrical conductivity, an oxidation-reduction potential, turbidity, and/or the like.

The illustrative analyte detection device 30 shown in FIGS. 3A and 3B is not intended to suggest any limitation as to the scope of use or functionality of embodiments of the present disclosure. The illustrative analyte detection device 30 also should not be interpreted as having any dependency or requirement related to any single component or combination of components illustrated therein. Additionally, various components depicted in FIGS. 3A and 3B may be, in embodiments, integrated with various ones of the other components depicted therein (and/or components not illustrated), all of which are considered to be within the ambit of the present disclosure.

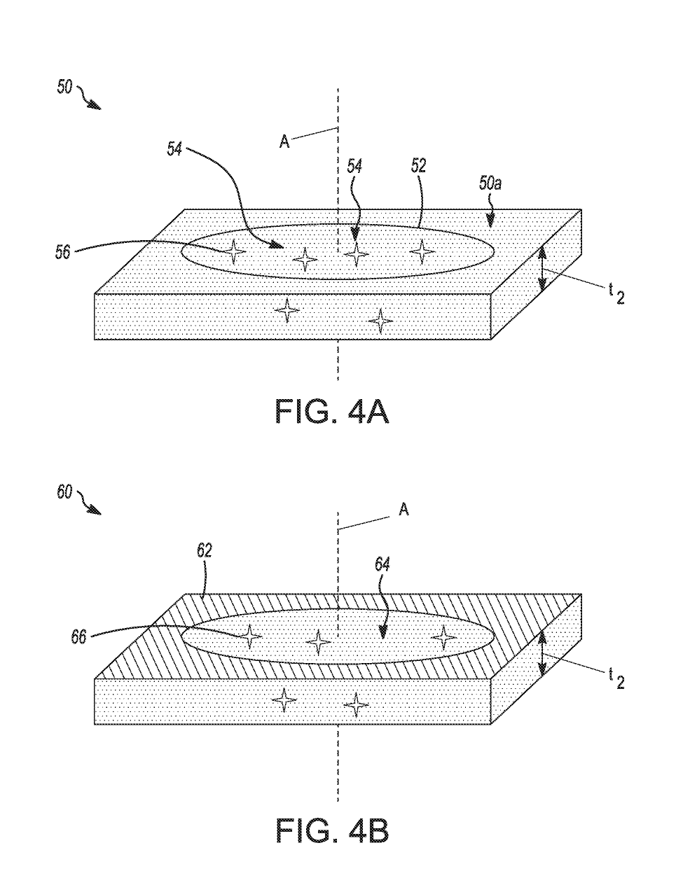

FIG. 4A depicts an illustrative perspective view of a reagent-hosting layer 50 having an optional barrier 52, in accordance with embodiments of the subject matter disclosed herein. In embodiments, the reagent-hosting layer 50 may be, be similar to, include, or be included in the reagent-hosting layer 16 depicted in FIG. 2, and/or the reagent-hosting layer 34 depicted in FIGS. 3A and 3B. As shown, the reagent-hosting layer 50 may include a detection zone 54. The detection zone 54 may be at least partially defined by the barrier 52. That is, for example, the detection zone 54 may be defined within a circumference and the barrier 52 may be disposed along the circumference. The barrier 52 may extend at least partially through the thickness t₂ of the reagent-hosting layer 50. In embodiments, the barrier 52 may be disposed on an upper surface 50A of the reagent-hosting layer 50. In embodiments, the barrier 52 may include any number of different shapes, and, in embodiments, the reagent-hosting layer 50 may include multiple barriers 52, while, in other embodiments, the reagent-hosting layer 50 may not include any barriers (other than the edges of the layer 50).

In embodiments, the barrier 52 may be configured to impede fluid transport in the device, particularly in the reagent-hosting layer 50, containing a volume of sampling fluid within the detection zone 54. One or more barriers may provide a complete impediment to fluid flow or, in embodiments, may reduce the flow rate of sampling fluid flowing radially through the reagent-hosting layer 50. In embodiments, the barrier 52 may serve as a physical and/or chemical barrier that may inhibit flow of sampling fluid outwardly beyond the barrier 52 and/or beyond the detection zone 54. In embodiments, the barrier 52 may serve as a physical and/or chemical barrier that may inhibit impregnation (or otherwise) of one or more reagents 56 beyond the detection zone 54. As shown in FIG. 4B, which depicts an illustrative perspective view of a reagent-hosting layer 60 having a barrier 62, in accordance with embodiments of the subject matter disclosed herein, the barrier 62 extends at least partially to the edges of the reagent-hosting layer 60. The barrier 62 may be similar to and/or have similar properties and/or functions as the barrier 52 depicted in FIG. 4A and described above. That is, for example, the barrier 62 may serve as a physical or chemical barrier for inhibiting flow of sampling fluid and/or beyond a detection zone 64, for inhibiting impregnation of reagents 66, and/or the like. As shown in FIGS. 4A and 4B, the barriers 52 and 62 may be disposed about a central axis A. In embodiments, the reagent-hosting layer 60 may be, be similar to, include, or be included in the reagent-hosting layer 16 depicted in FIG. 2, the reagent-hosting layer 34 depicted in FIGS. 3A and 3B, and/or the reagent-hosting layer 50 depicted in FIG. 4A.

In embodiments, one or more of the barriers 52 and 62 may be disposed along a circumference of the detection zone, where the barrier is continuous or non-continuous. The barrier geometry may take the form of any advantageous shape that benefits device performance. The barrier may only partially bind the lateral flow of fluid. The barrier may be semi-permeable, which may reduce the rate of fluid flow but not completely cease it. Examples of barrier materials used in the porous medium include at least one material chosen from a polymer, a glass, a ceramic, a wax, a paint, a cured resin, a plastic, a rubber, a thin film, a metal, metal oxide materials, solder or solder paste, a plurality of microparticles, a plurality of nanoparticles, and combinations thereof. Using particles as a non-continuous barrier includes the particles filling pores in the reagent-hosting layer 16 to create a barrier to flow. Alternatively, another means of creating a barrier to flow is to remove or partially remove the porous medium of layer 16 itself in order to restrict flow.

In embodiments, the barrier 52, for example, serves as an optical or visual tool for identifying the detection zone and may aid in image analysis. For example, in embodiments, an image analysis application such as embodiments discussed above may be configured to provide, via a user interface, a virtual barrier, which represents an outer border of a detection zone, and which may be aligned, using a camera, with an actual barrier on a device 10 so as to facilitate image analysis.

The illustrative reagent-hosting layers 50 and 60 shown in FIGS. 4A and 4B, respectively, are not intended to suggest any limitation as to the scope of use or functionality of embodiments of the present disclosure. The illustrative reagent-hosting layers 50 and 60 also should not be interpreted as having any dependency or requirement related to any single component or combination of components illustrated therein. Additionally, various components depicted in FIGS. 4A and 4B may be, in embodiments, integrated with various ones of the other components depicted therein (and/or components not illustrated), all of which are considered to be within the ambit of the present disclosure.

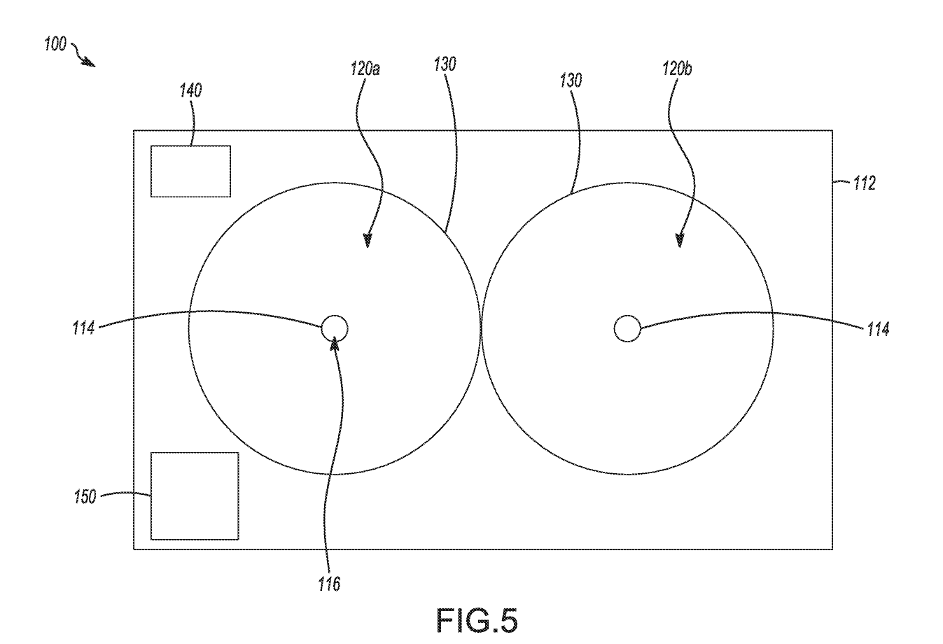

FIG. 5 depicts an illustrative top view of an analyte detection device card 100 having more than one detection zone for analysis, in accordance with embodiments of the subject matter disclosed herein. Analyte detection device card 100, interchangeably referred to herein as device 100 or card 100, includes two detection zones 120a and 120b, although embodiments may include any number of detection zones (e.g., one or more, including more than two). The device 100 includes at least two layers, including a top layer 112 having at least one inlet port 114 and a reagent-hosting layer 116 disposed adjacent to the top layer 112. In embodiments, the device 100 includes an inlet port 114 corresponding to each detection zone 120a and 120b. In embodiments, the inlet ports 114 may be concentrically disposed about individual axes (not shown). In embodiments, the device 100 and/or any of its components may be, be similar to, include, or be included within the device 10 (and/or any of its components) depicted in FIGS. 1 and 2; the device 30 (and/or any of its components) depicted in FIGS. 3A and 3B; the reagent-hosting layer 50 (and/or any of its components) depicted in FIG. 4A; and/or the reagent-hosting layer 60 (and/or any of its components) depicted in FIG. 4B.

According to embodiments, the top layer 112 may be impermeable, and the reagent-hosting layer 116 may be porous and impregnated, or otherwise loaded, with chemical and/or bio-chemical reagents. The device 100 includes detection zones 120a and 120b; and the reagents disposed within the detection zone 120a may be the same as, or different than, the reagents disposed within the detection zone 120b. The device 100 may optionally include at least one additional layer, e.g., a support layer (e.g., the support layer 18 depicted in FIG. 2), which may be impermeable. The detection zones 120a and 120b may be visually delineated or otherwise indicated by barriers 130, which may be similar to, and/or function as, the barriers 52 depicted in FIG. 4A and/or the barrier 62 depicted in FIG. 4B. As shown, the barriers 130 may be concentrically disposed about individual axes (e.g., the axis A depicted in FIG. 3A, the axis A depicted in FIG. 3B, the axis A depicted in FIG. 4A, the axis A depicted in FIG. 4B). In embodiments, the card 100 may include information to aid a user of the device to obtain and analyze data quickly. In embodiments, for example, the card 100 includes information (numeric, alphabetic, or symbolic) to identify the compound or element to be quantified, for example, the element ‘Fe’ and associated data may be included in an identifier 140 disposed on the card 100. The identifier 140 may be located any place that the user can readily see. The card 100 may further include a ‘QR’ code 150 (or barcode or other similar feature) to enable the user to quickly access further information for obtaining and/or for reading results. In embodiments, the location of the QR code 150 may be any place that the user can readily see or read with a smart phone or device.

The illustrative analyte detection device card 100 shown in FIG. 5 is not intended to suggest any limitation as to the scope of use or functionality of embodiments of the present disclosure. The illustrative analyte detection device card 100 also should not be interpreted as having any dependency or requirement related to any single component or combination of components illustrated therein. Additionally, various components depicted in FIG. 5 may be, in embodiments, integrated with various ones of the other components depicted therein (and/or components not illustrated), all of which are considered to be within the ambit of the present disclosure.

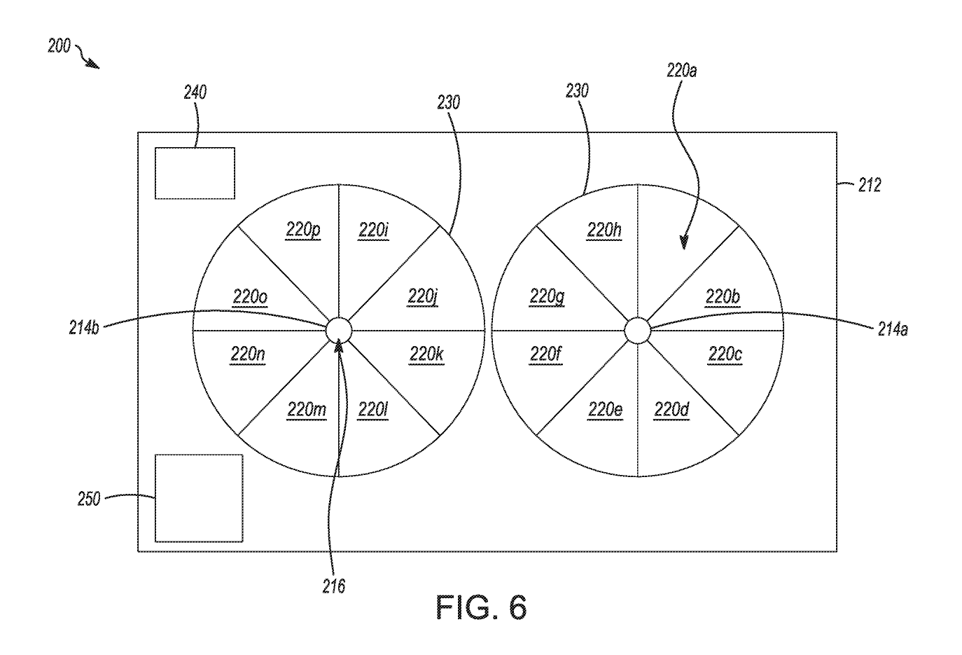

FIG. 6 depicts an illustrative top view of an analyte detection device card 200 having multiple detection zones for analysis, in accordance with embodiments of the subject matter disclosed herein. The analyte detection device card 200, interchangeably referred to herein as device 200 or card 200, includes a number of detection zones. In the illustrated example, the card 200 includes sixteen detection zones 220a-220p. In embodiments, the card 200 may include any number of detection zones. The detection zones 220a-220p may be loaded with reagents. In embodiments, each zone 220a-220p is loaded with a different reagent configured to react with different analytes in sampling fluid introduced via inlet ports 214a and 214b. In embodiments, the device 200 and/or any of its components may be, be similar to, include, or be included within the device 10 (and/or any of its components) depicted in FIGS. 1 and 2; the device 30 (and/or any of its components) depicted in FIGS. 3A and 3B; the reagent-hosting layer 50 (and/or any of its components) depicted in FIG. 4A; the reagent-hosting layer 60 (and/or any of its components) depicted in FIG. 4B, and/or the device 100 depicted in FIG. 5.

According to embodiments, the device 200 includes at least two layers, including a top layer 212 having at least one inlet port and a reagent-hosting layer 216 disposed adjacent to the top layer 212. The inlet ports 214a and 214b may be concentrically disposed about individual axes (e.g., the axis A depicted in FIG. 3A, the axis A depicted in FIG. 3B, the axis A depicted in FIG. 4A, the axis A depicted in FIG. 4B) and may facilitate providing sampling fluid to one or more detection zones. As shown in FIG. 6, the inlet port 214a provides sampling fluid to the detection zones 220a-220h, and the inlet port 214b provides sampling fluid to the detection zones 220i-220p. In embodiments, the top layer 212 may be impermeable; and the reagent-hosting layer 216 may be porous and impregnated, or otherwise loaded, with chemical and/or bio-chemical reagents. In embodiments, the device 200 may include at least one additional layer, e.g., a support layer, which may be impermeable. The outer perimeter of detection zones 220a-220p may be visually delineated or otherwise indicated by barriers 230, which may be similar to, and/or function as, the barriers 52 depicted in FIG. 4A, the barrier 62 depicted in FIG. 4B, and/or the barriers 130 depicted in FIG. 5. As with inlet ports 214a and 214b, the barriers 230 may be concentrically disposed about individual axes (e.g., the axis A depicted in FIG. 3A, the axis A depicted in FIG. 3B, the axis A depicted in FIG. 4A, the axis A depicted in FIG. 4B). In embodiments, the card 200 optionally includes information to aid a user of the device to obtain and analyze date quickly. In embodiments, card 200 includes information (numeric, alphabetic, or symbolic) to identify the compound or element to be quantified, for example, the element ‘Fe’ and associated data is included in an identifier 240 on card 200. The identifier 240 on card 200 may be located any place that the user can readily see. Card 200 may further include a ‘QR’ code 250 (or other similar feature) to enable the user to quickly access further information for obtaining and/or for reading results; and the location of the QR code 250 may be any place that the user can readily see or read with a smart phone or device. In embodiments, for example, a mobile application may be configured to receive an image, from an imaging device of a mobile device (e.g., a smartphone camera), of the QR code 250 (or other similar feature) and determine, based on the QR code 250 (or other similar feature), the analyte (or type of analyte) that the card 200 is configured to be used for testing in association therewith.

According to embodiments, devices described herein may facilitate determination of analytes in sampling fluid. Non-limiting examples of analytes include at least one analyte chosen from a proton concentration (i.e. pH value), a metal concentration (e.g. Calcium, Magnesium, Aluminum, Iron, Lead, Zinc, Copper, Cadmium, Manganese, Chromium, Nickel), a non-metallic inorganic species (e.g. Chloride, Chlorite, Hypochlorite, Nitrate, Nitrite, Ammonia), an organic species (e.g. Ethanol, Glucose, Acetic Acid, Pesticides, Hormones), and a biochemical compounds (e.g. enzymes, enzyme substrates). While the preceding analytes are presented as illustrative examples, the listing is not all-encompassing of the species or properties that may be detected using embodiments of the device or method of the present disclosure.

The device may include an inlet port that restricts the flow of fluid into the device. Fluid may be driven through the porous medium by capillary action and/or by using a positive or negative pressure to actively drive fluid through the medium. Fluid may be delivered to the device using a vial or pipette. Alternatively, fluid may be delivered without restricting the fluid volume by dropping fluid on to the top of the inlet. Methods of using a timer also may be used to create a consistent fluid volume delivered to the porous medium by removing excess fluid once a set time has expired. Alternatively or in addition to, a barrier within layer 16 allows fluid delivery of a fixed volume to the device.

As fluid enters the device, the analytes present in the fluid encounter the reagents supported by the reagent-hosting layer to provide an indicator. The analytes react with the reagents yielding a detectable change and/or visual indicator as detailed above. As the analytes encounter and react with the reagents loaded in the reagent-hosting layer, they may be largely removed from the fluid. Once the analyte has been largely removed from the fluid, the fluid may continue to propagate radially from the inlet disposed about axis A but without a detectable change as described above. The termination of the detectable change serves as an endpoint of the reaction between the reagent(s) and analyte(s). The termination of the detectable change is measurable (e.g., radial distance or diameter).

To quantify the analyte(s) present in the fluid the geometric properties of the detectable change may be measured. In embodiments where a circular inlet guides fluid to a porous medium with a colorimetric indicator reagent that is unbounded in the plane of the porous medium, a largely circular pattern develops. Once the fluid has ceased to flow, the area (or diameter or radius or geometric derivations of those parameters) of the color formed may be related to the concentration of the analyte in the fluid. Typically, a calibration curve is constructed by delivering fluids with known concentration of analytes to individual devices. The geometric properties of the area where analyte has reacted with reagents can be quantified a number of different ways. A ruler, or other geometric measurement tool, may be used to measure the geometric dimensions. Additionally, images of the device may be analyzed using analysis algorithms based on the detectable changes. The algorithms may be executed by computers (e.g., smartphones, tablets, workstations, laptops, etc.) or conducted by users.

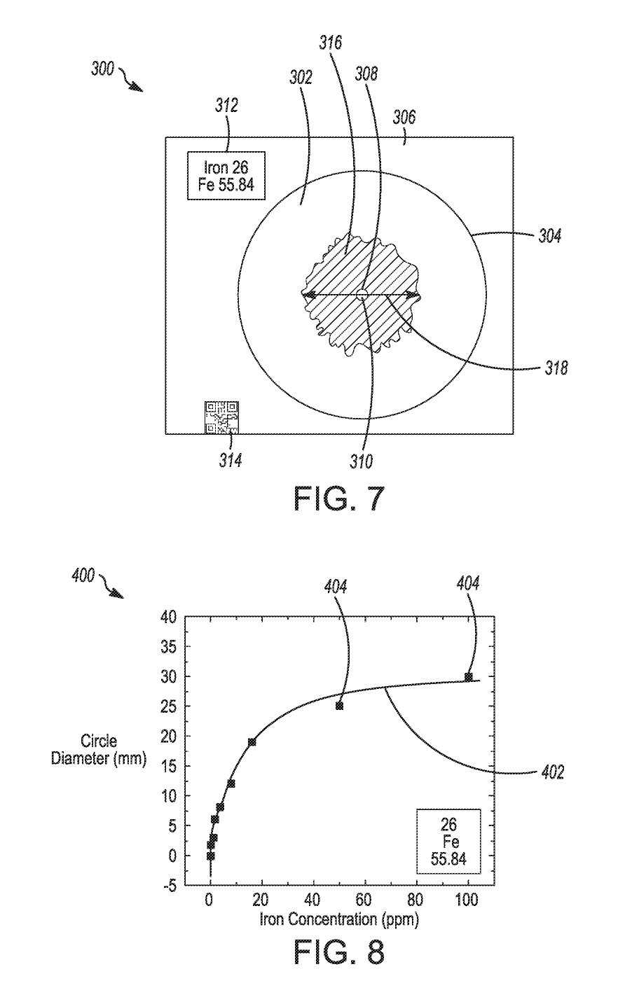

FIG. 7 depicts an illustrative top view of an analyte detection device card 300 configured to facilitate determining a concentration of iron in a sampling fluid, and having a detection zone 302 bounded by a barrier 304, in accordance with embodiments of the subject matter disclosed herein. The analyte detection device card 300 (referred to, interchangeably, as a device and/or a card) includes at least two layers, including a top layer 306 having at least one inlet port 308 and a reagent-hosting layer 310 disposed adjacent to the top layer 306. In embodiments, the card 300 and/or any of its components may be, be similar to, include, or be included within the device 10 (and/or any of its components) depicted in FIGS. 1 and 2; the device 30 (and/or any of its components) depicted in FIGS. 3A and 3B; the reagent-hosting layer 50 (and/or any of its components) depicted in FIG. 4A; the reagent-hosting layer 60 (and/or any of its components) depicted in FIG. 4B; the analyte device detection card 100 (and/or any of its components) depicted in FIG. 5, and/or the analyte detection device card 200 (and/or any of its components) depicted in FIG. 6.

According to embodiments, the top layer 306 may be impermeable, and the reagent-hosting layer 310 may be porous and impregnated, or otherwise loaded, with chemical and/or bio-chemical reagents. That is, for example, embodiments of the reagent-hosting layer 310 may be loaded with a chemical reagent configured to react with iron in a sampling fluid, thereby indicating a characteristic (e.g., a presence and/or concentration) of iron in the sampling fluid. The card 300 may optionally include at least one additional layer, e.g., a support layer (e.g., the support layer 18 depicted in FIG. 2), which may be impermeable. As shown, the card 300 includes an identifier 312 indicating that the card 300 is configured for determining a characteristic of iron. The card 300 may further include a ‘QR’ code 314 to enable the user to quickly access further information for obtaining and/or for reading results.

In the illustrated embodiments of FIG. 7, a sampling fluid (not shown) has been introduced, via the inlet port 308, to the reagent-hosting layer 310, and iron within the sampling fluid has reacted with the reagent loaded on the reagent-hosting layer 310, causing a region 316 of visible color change. As described above, embodiments may facilitate measuring a diameter 318 of the region 316 of color change and determining a concentration of iron within the sampling fluid based on a mathematical relationship between the diameter 318 and a concentration of iron in the type of sampling fluid that was tested. In the illustrated embodiment, the test has indicated that the sampling fluid comprises approximately a 10 parts per million (ppm) total iron solution.

According to embodiments, the diameter 318 may refer to a single diameter measurement, multiple diameter measurements, an aggregation of multiple diameter measurements (e.g., an average diameter measurement, a normalized diameter measurement), and/or the like (any or all of which may be referred to simply as “a diameter” or “diameter measurement” or similar, in various embodiments). In embodiments, the concentration of iron may be determined by comparing the diameter to the response of known standards, which may be embodied, for example, in a table, a graph, a curve, a chart, and/or the like. For example, FIG. 8 depicts an illustrative graph 400 showing a calibration curve 402 assembled by testing a series of reference solutions to obtain a set of sample points 404 representing the corresponding diameters or color change regions at various iron concentrations, in accordance with embodiments of the subject matter disclosed herein. The calibration curve 402 may be used to determine the iron concentration of an unknown sample. According to embodiments, the calibration curve may be represented as a mathematical relation that may be used, by a human and/or a computing device, to determine the iron concentration of a sampling fluid. Though the illustrative calibration curve 402 is described herein in terms of iron concentration, the same, or similar, calibration curve concepts may be utilized with regard to any number of different characteristics of any number of different types of analytes.

In operation, for example, a user may introduce a sampling fluid to the inlet port 308 of the card 300, which may result in a reaction that creates the region 316 of color change. In embodiments, the user may measure a diameter 318 of the region 316 using a ruler or other manual instrument. In embodiments, the user may instantiate an application on a computing device (e.g., a mobile device such as a smartphone) and use the device to acquire an image of the QR code 314. The application may be configured to receive the image of the QR code 314 from the imaging device and to determine, based on the image of the QR code 314, that the card 300 is configured to facilitate determining a concentration of iron in a sampling fluid. The application may be configured to, in response to determining the analyte associated with the card 300 (and/or the type of characteristic to be determined), access a calibration curve associated with iron. The application may, via any number of different image analysis techniques, determine a diameter of the region 316 of color change, and to compare the diameter to the calibration curve to determine the concentration of iron in the sampling fluid. In response to making this determination, in embodiments, the application may be configured to provide a representation of the concentration to a user such as, for example, by displaying the concentration on a graphical user interface (GUI). In embodiments, the application may be configured to store the determined concentration, use the determined concentration to determine some other characteristic of the iron and/or sampling fluid, transmit the determined concentration (and/or information associated therewith) to another device (e.g., another mobile device, a server, etc.), and/or the like.

The illustrative analyte detection device card 300 and graph 400 shown in FIGS. 7 and 8, respectively, are not intended to suggest any limitation as to the scope of use or functionality of embodiments of the present disclosure. The illustrative analyte detection device card 300 and graph 400 also should not be interpreted as having any dependency or requirement related to any single component or combination of components illustrated therein. Additionally, various components depicted in FIGS. 7 and 8 may be, in embodiments, integrated with various ones of the other components depicted therein (and/or components not illustrated), all of which are considered to be within the ambit of the present disclosure.

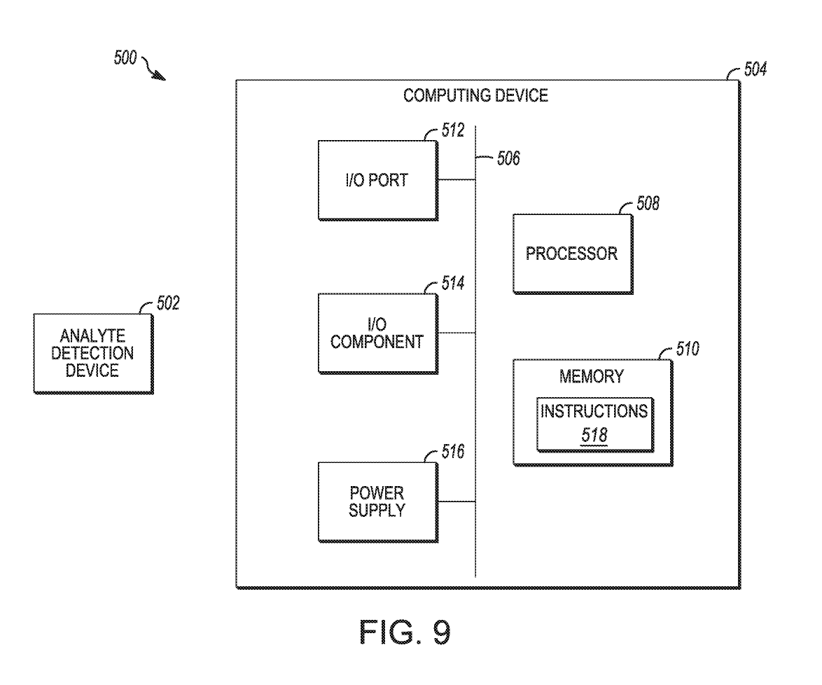

FIG. 9 is a block diagram depicted an illustrative analyte detection system 500, in accordance with embodiments of the subject matter disclosed herein. As shown, the system 500 may include an analyte detection device 502. The analyte detection device may be configured to receive a sampling fluid and to produce an indicator of a characteristic of an analyte in the sampling fluid and/or of the sampling fluid, as described herein. In embodiments, the analyte detection device 502 may be, be similar to, include, or be included within the device 10 (and/or any of its components) depicted in FIGS. 1 and 2; the device 30 (and/or any of its components) depicted in FIGS. 3A and 3B; the reagent-hosting layer 50 (and/or any of its components) depicted in FIG. 4A; the reagent-hosting layer 60 (and/or any of its components) depicted in FIG. 4B; the analyte device detection card 100 (and/or any of its components) depicted in FIG. 5; the analyte detection device card 200 (and/or any of its components) depicted in FIG. 6; and/or the analyte detection device card 300 (and/or any of its components) depicted in FIG. 7.

The system 500 may further include a computing device 504 configured to obtain an image of a detection zone of the analyte detection device 502, determine a diameter of a region of a color change within the detection zone, and determine, based on the diameter, one or more characteristics of the analyte and/or the sampling fluid, as described in association with embodiments described herein. The computing device 504 may include any type of computing device suitable for implementing aspects of embodiments of the disclosed subject matter. Examples of computing devices include specialized computing devices or general-purpose computing devices such “workstations,” “servers,” “laptops,” “desktops,” “tablet computers,” “hand-held devices,” “smartphones,” “general-purpose graphics processing units (GPGPUs),” and the like, all of which are contemplated within the scope of FIG. 8.

In embodiments, the computing device 504 includes a bus 506 that, directly and/or indirectly, couples the following devices: a processor 508, a memory 510, an input/output (I/O) port 512, an I/O component 514, and a power supply 516. Any number of additional components, different components, and/or combinations of components may also be included in the computing device 504. The I/O component 514 may include a presentation component configured to present information to a user such as, for example, a display device, a speaker, a printing device, and/or the like, and/or an input component such as, for example, a microphone, a joystick, a satellite dish, a scanner, a printer, a wireless device, a keyboard, a pen, a voice input device, a touch input device, a touch-screen device, an interactive display device, a mouse, and/or the like.

The bus 506 represents what may be one or more busses (such as, for example, an address bus, data bus, or combination thereof). Similarly, in embodiments, the computing device 504 may include a number of processors 508, a number of memory components 510, a number of I/O ports 512, a number of I/O components 514, and/or a number of power supplies 516. Additionally any number of these components, or combinations thereof, may be distributed and/or duplicated across a number of computing devices.

In embodiments, the memory 510 includes computer-readable media in the form of volatile and/or nonvolatile memory and may be removable, nonremovable, or a combination thereof. Media examples include Random Access Memory (RAM); Read Only Memory (ROM); Electronically Erasable Programmable Read Only Memory (EEPROM); flash memory; optical or holographic media; magnetic cassettes, magnetic tape, magnetic disk storage or other magnetic storage devices; data transmissions; and/or any other medium that can be used to store information and can be accessed by a computing device such as, for example, quantum state memory, and/or the like. In embodiments, the memory 510 stores computer-executable instructions 518 for causing the processor 508 to implement aspects of embodiments of system components discussed herein and/or to perform aspects of embodiments of methods and procedures discussed herein.

The computer-executable instructions 518 may include, for example, computer code, machine-useable instructions, and the like such as, for example, program components capable of being executed by one or more processors 508 associated with the computing device 504. For example, in embodiments, the computer-executable instructions 518 may be configured to cause the one or more processors 508 to instantiate an application (e.g., “mobile app”) configured to facilitate any number of different aspects of analyte detection and/or analysis, as discussed throughout this document. Program components may be programmed using any number of different programming environments, including various languages, development kits, frameworks, and/or the like. Some or all of the functionality contemplated herein may also, or alternatively, be implemented in hardware and/or firmware.

The illustrative system 500 shown in FIG. 9 is not intended to suggest any limitation as to the scope of use or functionality of embodiments of the present disclosure. The illustrative system 500 also should not be interpreted as having any dependency or requirement related to any single component or combination of components illustrated therein. Additionally, various components depicted in FIG. 9 may be, in embodiments, integrated with various ones of the other components depicted therein (and/or components not illustrated), all of which are considered to be within the ambit of the present disclosure.

Various modifications and additions can be made to the exemplary embodiments discussed without departing from the scope of the presently disclosed subject matter. For example, while the embodiments described above refer to particular features, the scope of this disclosure also includes embodiments having different combinations of features and embodiments that do not include all of the described features. Accordingly, the scope of the subject matter disclosed herein is intended to embrace all such alternatives, modifications, and variations as fall within the scope of the claims, together with all equivalents thereof.

The following is claimed:

1. An analyte detection device, comprising:
a fluid impermeable layer having a first thickness and comprising at least one inlet port having a diameter, the at least one inlet port defining a fluid pathway through the first thickness; and
a reagent-hosting layer having a second thickness and comprising at least one of a chemical reagent and a bio-chemical reagent, the reagent-hosting layer configured to radially receive a sampling fluid via the fluid pathway, the sampling fluid configured to interact with the at least one of a chemical reagent and a bio-chemical reagent in the porous layer to indicate a characteristic associated with an analyte in the sampling fluid.

2. The analyte detection device of claim 1, wherein the characteristic is at least one chosen from a chemical species, a biochemical species, a polarity, a refractive index, an oxidation-reduction potential, a redox activity, a turbidity, a pH, and combinations thereof.

3. The analyte detection device of claim 1, wherein the characteristic is the presence or absence of the analyte in the sampling fluid.

4. The analyte detection device of claim 1, wherein the diameter is greater than or equal to approximately 0.5 mm and less than or equal to approximately 10 mm.

5. The analyte detection device of claim 1, wherein the second thickness is greater than or equal to approximately 0.05 mm and less than or equal to approximately 2.0 mm.

6. The analyte detection device of claim 4, wherein the sum of the first thickness and the second thickness is less than or equal to approximately 3.5 mm.

7. The analyte detection device of claim 1, the porous layer further comprising a detection zone, the detection zone having an additional diameter from greater than or equal to approximately 1 mm and less than or equal to approximately 500 mm.

8. The analyte detection device of claim 7, the detection zone indicating the characteristic of the analyte via a color change.

9. The analyte detection device of claim 7, further comprising a central axis wherein the inlet port and the detection zone are concentric about the central axis.

10. The analyte detection device of claim 7, further comprising a barrier along a circumference of the detection zone, wherein the barrier is continuous or non-continuous.

11. The analyte detection device of claim 10, wherein the barrier comprises at least one of a polymer, a glass, a ceramic, a wax, a paint, a cured resin, a plastic, a rubber, a thin film, a metal, a plurality of microparticles, and a plurality of nanoparticles.

12. The analyte detection device of claim 1, further comprising at least one additional layer, the additional layer having a third thickness, wherein the reagent-hosting layer is disposed between the fluid impermeable layer and the at least one additional layer.

13. The analyte detection device of claim 1, wherein the reagent-hosting layer comprises at least one of a porous medium and a fibrous medium.

14. The analyte detection device of claim 13, wherein the reagent-hosting layer comprises at least one of a paper, a filter paper, a nitrocellulose, a glass fiber mesh, a metal screen, a metal wool, a polymer coated fiber mesh, a polymer filter media, a woven graphite, a non-woven graphite, a carbon fiber mesh, a natural textile, a synthetic textile, a cotton, a wool, and a polyester.

15. The analyte detection device of claim 1, wherein the fluid impermeable layer is at least partially transparent.

16. The analyte detection device of claim 15, wherein the fluid impermeable layer comprises at least one of a polymer, a glass, a ceramic, a wax, a paint, a cured resin, a plastic encasement, a thin film, and a metal coating.

17. The analyte detection device of claim 1, wherein the at least one of a chemical reagent and a bio-chemical reagent in the porous layer is a material chosen from a redox dye, a solvent polarity dye, a Polypyridyl species, a plurality of nanoparticulates, a thiocarbazone, a glyoxime, a complexometric dye, a pH indicator dye, an Azo dye, and combinations thereof.

18. An analyte detection laminate, comprising:
a central axis;
a first layer perpendicular to the central axis and having an inlet aperture, the inlet aperture disposed about the central axis and defining a fluid pathway through the first layer;
a second layer, adjacent to the first layer and in fluid communication with the fluid pathway, the second layer comprising:
a reagent-hosting medium;
at least one of a chemical reagent and a bio-chemical reagent disposed in or on the host medium and configured to interact with a sampling fluid received via the fluid pathway to provide an indicator of a characteristic of an analyte in the sampling fluid;
a detection zone, extending radially from the central axis, for quantifying the indicator; and
a third layer adjacent to the second layer, wherein the second layer is porous relative to the first and third layers.

19. A method of making an analyte detection device, the method comprising:
forming a fluid impermeable layer having at least one inlet port defining a fluid pathway therethrough, the at least one inlet port configured to receive a sampling fluid;
laminating a reagent-hosting layer to the fluid impermeable layer, the porous layer in fluid communication with the at least one inlet port, the porous layer further comprising a detection zone; and
impregnating the detection zone with at least one of a chemical reagent and a bio-chemical reagent, wherein the at least one of a chemical reagent and a bio-chemical reagent is configured to interact with the sampling fluid received at the inlet port and flowing radially in the detection zone to provide an indicator of a characteristic of an analyte in the sampling fluid.

20. The method of claim 19, further comprising quantifying the indicator.
